(19) United States
(12) Patent Application Publication
(10) Pub. No.: US 2004/0267338 A1
(43) Pub. Date: Dec. 30, 2004

(54) Targeted tissue cooling within a body

(76) Inventor: Kent Harrison, Maple Grove, MN (US)

Correspondence Address: FISH & RICHARDSON PC, 225 FRANKLIN ST, BOSTON, MA, 02110, US

(21) Appl. No.: 10/603,705

(22) Filed: Jun. 25, 2003

(51) Int. Cl. A61F 7/00; A61F 7/12

(52) U.S. Cl. 607/105; 607/113

(57) ABSTRACT

A medical device has a structure deployable from its distal end to cool a target tissue region inside the body, such as tissue within a chamber of the heart. Also, a method of cooling a target tissue region inside a body includes introducing a distal portion of a catheter having a deployable structure at a distal end into the body. The method also includes positioning the distal catheter portion near a target tissue region, deploying the deployable structure, and placing the deployed structure in contact with the target tissue region to cool the target tissue region.

### TECHNICAL FIELD

The invention relates to cooling tissue inside a body.

### BACKGROUND

Myocardial ischemia, and in severe cases an acute myocardial infarction (AMI), can occur when there is inadequate blood circulation to the myocardium due to coronary artery disease. Evidence suggests that early reperfusion of blood into the heart, after removing a blockage to blood flow, dramatically reduces damage to the myocardium. However, the reestablishment of blood flow into the heart may cause a reperfusion injury to occur. Reperfusion injury is believed to be due to the build up of waste products on the myocardium during the time blood flow was inadequate and the reaction of these waste products with oxygen in the blood when normal blood flow is reestablished.

It is possible to reduce reperfusion injury to the myocardium by cooling the myocardial tissue prior to reperfusion. Mild cooling of the myocardial tissue to a temperature of 33 degrees Celsius, which is approximately four degrees cooler than normal body temperature, provides a protective effect, likely by the reduction in the rate of chemical reactions and the reduction of tissue activity and associated metabolic demands. Although 33 degrees Celsius is the target cooling temperature, cooling between 28 to 36 degrees Celsius may also be beneficial.

One way to cool myocardial tissue is by placing an ice pack over the patient's heart. Another method involves puncturing the pericardium and providing cooled fluid to a reservoir inserted into the pericardial space near the targeted myocardial tissue. Cooling of the myocardial tissue may also be accomplished by perfusing the target tissue with cooled solutions. A catheter having a heat transfer element located in the catheter's distal tip may also be inserted into a blood vessel to cool blood flowing into and through the heart. It is also possible to cool the myocardial tissue by supplying cool blood to the heart through a catheter placed in the patient's coronary sinus.

### SUMMARY

The invention provides devices and methods to cool a target tissue region inside the body, for example within a chamber of the heart. In one aspect, the invention features a medical device including an elongate body having a distal end for entry into a body and positionable near a target tissue region within the body. The medical device also has a structure deployable from the distal end of the elongate body to cool the target tissue region is provided.

Various embodiments may include one or more of the following features. The elongate body of the medical device may include an elongate shaft that has the deployable structure affixed to its distal end. The body of the medical device may also include an elongate sleeve that is longitudinally moveable with respect to the shaft and that when moved distally encompasses the deployable structure. The distal end of the device may be advanceable through a body vessel to the target tissue region when the structure is in a non-deployed state but may not be advancable through the body vessel when the structure is in a deployed state. The elongate body may further include a proximal end that remains outside the body when the distal end of the elongate body is positioned near the target tissue region.

In addition, the deployable structure may include a patch having a surface shaped to contact the tissue region and an inner chamber that receives fluid from a lumen in the elongate body for cooling the patch surface that contacts the tissue region. The deployable structure may also be cup-shaped and include a periphery for contacting body tissue to form a chamber whose bounds are defined by the body tissue and the inside surface of the cup-shaped structure. In implementations, the deployable structure may have an inner chamber with a Joule-Thompson orifice into the inner chamber so that a liquid supplied through the elongate body, through the orifice, and into the inner chamber has a phase change into a gas.

In another aspect, the invention features a medical device with an elongate body having a distal end for entry into a body and positionable near a target tissue region within the body. The device has a patch deployable from the distal end of the elongate shaft to cool the target tissue region. The patch has a surface shaped to contact the target tissue region.

In addition, the patch may include a collapsible frame made of a shape memory alloy so that, when deployed, the patch expands to create the surface that contacts the target tissue region. The elongate shaft may include a first lumen to provide fluid to an inner chamber of the patch and a second lumen to remove fluid from the inner chamber of the patch. The inner chamber of the patch can include a conduit through which fluid flows, the conduit being located adjacent to the surface of the patch in contact with the target tissue region. In implementations, the medical device may further include at least one additional patch deployable from the distal end of the elongate body.

The patch may also include a thermoelectric cooling element positioned adjacent to the surface of the patch and in contact with the target tissue region to cool the tissue region. In other implementations, the patch may have an inner chamber with a Joule-Thompson orifice into the inner chamber so that a liquid supplied through the elongate shaft, through the orifice, and into the inner chamber has a phase change into a gas.

The medical device may further include a balloon positioned adjacent to a surface of the patch that does not contact the target tissue region. The balloon may provide insulation between the patch and body fluids when the patch is deployed and positioned near the target tissue region. A lumen in the elongate body may be included to provide the balloon with an inflation medium. The medical device can also have an anchoring mechanism near the distal end of the elongate shaft that is connectable to tissues inside the body to anchor the patch when it is deployed near the target tissue area. A temperature sensor to sense the temperature of the surface of the patch in contact with the target tissue region may also be included.

In another aspect, the invention features a medical device including an elongate body having fluid transfer lumens extending longitudinally through the shaft to a distal end. A cup-shaped structure deployable from the distal end of the body and having a periphery for contacting body tissue to form a chamber bound by the body tissue and an inside surface of the cup-shaped structure is also included. The chamber is in fluid communication with the body fluid transfer lumens to allow fluid to be delivered to and from the chamber.

Additionally, the cup-shaped structure may include a collapsible frame made of a shape memory alloy so that, when deployed, the cup-shaped structure can expand to take on a cup-shaped configuration. The periphery of the cup-shaped structure can have small holes positioned to contact the body tissue, the holes securing the periphery of the cup-shaped structure to the body tissue when a vacuum is applied. The cup-shaped structure may include a layered structure with an inflatable chamber positioned between first and second layers and inflatable to insulate body fluids from the fluid in the sealed chamber. The elongate shaft may have a lumen to provide the inflatable chamber with an inflation medium. The medical device may also include a temperature sensor to sense the temperature of the fluid provided to the chamber.

Implementations may include other features. For example, the elongate body of the medical devices may include an elongate shaft that has the deployable patch or cup-shaped structure affixed to its distal end and an elongate sleeve that is longitudinally movable with respect to the shaft. When the sleeve is moved distally, these structures are encompassed by the sleeve. The structures may be deployed from the distal end of the body by moving the sleeve proximally with respect to the shaft to expose the structures from the confines of the sleeve. In some implementations, the distal end of the device is advanceable through a body vessel to the tissue region when the structures are in a non-deployed state and not advanceable through the body vessel when the structures are in a deployed state.

In another aspect, the invention features a method of cooling a target tissue region inside a body. The method includes introducing into a body vessel a distal portion of a catheter having an elongate body and a structure deployable from a distal end of the elongate body. The distal portion of the catheter is positioned near the target tissue region and the deployable structure is deployed from the elongate body placing the deployed structure in contact with the target tissue region. The deployed structure is cooled to cool the target tissue region.

In addition, the deployable structure may include a patch having a surface shaped to contact the tissue region. The deployable structure may also be a cup-shaped and have a periphery for contacting to the body tissue region to form a chamber bound by the body tissue and an inside surface of the cup. The target tissue region may be within a chamber of the heart, in which case the deploying of the structure may occur after the distal end of the catheter is positioned inside the heart chamber.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a perspective view of a medical device that has a deployable patch for cooling a target tissue area inside the body.

FIG. 2 is a perspective view of the medical device shown in FIG. 1 showing the patch in a non-deployed state.

FIG. 3 is a top cross-sectional view of a distal portion of the medical device shown in FIG. 1.

FIG. 4 is a side view of the medical device shown in FIG. 1.

FIG. 5 shows a medical device placed inside a chamber of the heart to cool a target tissue region.

FIG. 6 is a perspective view of a medical device that has a deployable cup for cooling a target tissue region inside the body.

FIG. 7 is a cross-sectional view of a distal portion of the medical device shown in FIG. 6.

FIG. 8 is a bottom view of a distal portion of the medical device shown in FIG. 6.

FIG. 9 is a cross-sectional view of a distal portion of an alternative medical device that has a deployable structure for cooling a target tissue region inside the body.

FIG. 10 is a top view of a patch having a flow circuit.

FIG. 11 is a top view of a patch having a flow circuit.

FIG. 12A is a side cross-sectional view of a patch using thermoelectric cooling elements to cool a target tissue region.

FIG. 12B is a top view of a portion of the patch shown in FIG. 12A.

Like reference symbols in the various drawings indicate like elements.

### DETAILED DESCRIPTION

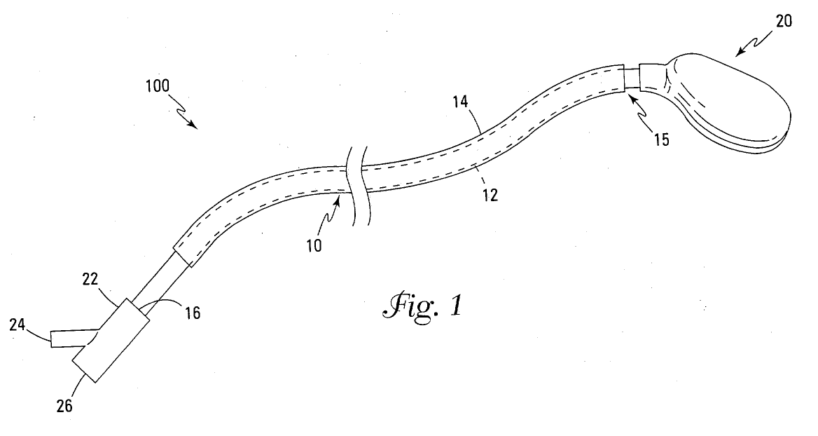

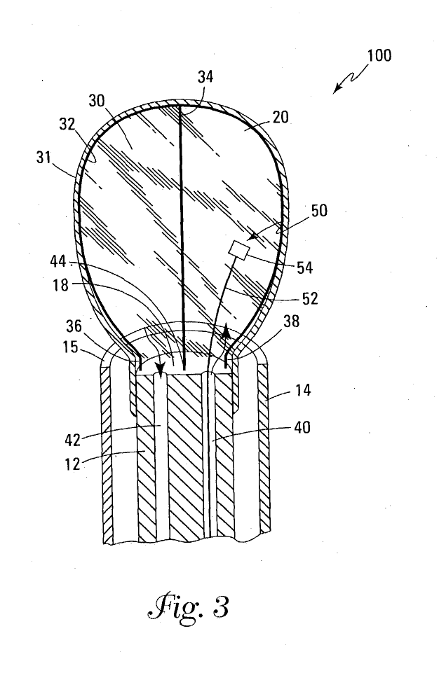

A medical device 100, shown in FIGS. 1-5, includes an elongate body 10 and a balloon-shaped patch 20 that is deployable from a distal end 15 of the body 10 to cool a targeted region of internal body tissue. FIG. 1 shows the entire device 100 in a deployed state. To cool the target tissue region, the device 100 is inserted into a body lumen, such as a blood vessel, in a non-deployed state (not shown in FIG. 1) and advanced until the device's distal end 15 is positioned near the target tissue region, such as inside a heart chamber. When the device's distal end 15 is so positioned, the patch 20 may be deployed from the elongate body 10 and positioned against the tissue to be cooled. Cooled fluid may then be introduced into the device 1100 and circulated through an internal chamber 30 (shown in FIG. 3) of the patch 20 to cool a surface of the patch 20 in contact with the tissue, and thus provide localized cooling of the contacted tissue region.

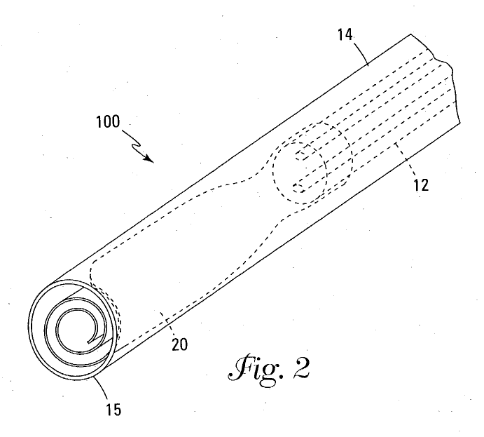

Referring to FIG. 1, the device's body 10 includes an elongate shaft 12 and an elongate sleeve 14 that encompasses the shaft 12: The patch 20 is attached to a distal end 18 of the shaft 12 (the distal end 18 being'shown in FIG. 3). The elongate sleeve 14 is movable longitudinally and independently with respect to the shaft 12. FIG. 1 shows the patch 20 in a deployed state where the sleeve 14 has been moved proximally with respect to the shaft 12 so as to reveal the patch 20 from the confines of the sleeve 14. To put the patch in the non-deployed state, the sleeve 14 may be pushed distally with respect to the shaft 12 so that the patch 20 collapses to fit within the sleeve 14. FIG. 2 shows a distal portion of the device 100 with the patch 20 collapsed and confined within the elongate sleeve 14. In the non-deployed state shown in FIG. 2, the device 100 may be advanced through vessels and other internal body regions where the advancement may not be possible with the patch 20 in the deployed state, given the size and shape of the patch 20 when deployed.

Referring again to FIG. 1, an adapter 22 is attached to a proximal end 16 of the shaft 12, and remains outside of the patient's body when the device 100 is in use. Two lumens 40 and 42 (distal portions of which are shown in the cross-sectional diagram of FIG. 3) extend longitudinally through the shaft 12 from the adapter 22 attached to the shaft's proximal end 16 to the patch 20 attached to the shaft's distal end 18. The cooled fluid mentioned previously may be introduced into the adapter 22 whereupon the fluid flows into a first lumen 40 of the shaft 12 and distally through that lumen 40 and into an internal chamber 30 of the patch 20 (as indicated by the arrow shown in FIG. 3). The cooled fluid then exits the patch internal chamber 30 and enters a second lumen 42. The fluid flows proximally through that lumen 42 and back to the adapter 22 shown in FIG. 1. The adapter 22 has two ports 24 and 26. The cooled fluid may be introduced into the device 100 via the first port 24 and exit out of the second port 26, or vice versa.

The fluid that is circulated through the internal chamber 30 of the patch 20 may be a liquid that changes phase into a gas by the Joule-Thomson effect when it enters the chamber. In some implementations, the first lumen 40 may include a Joule-Thompson orifice at its distal end so that liquid flowing through the lumen 40 changes into a gaseous state upon entry into the internal chamber 30, and the second lumen 42 functions to remove gas from the chamber 30. Alternatively, the fluid circulated may remain in a liquid state as it is circulated through the chamber. The circulation of cooled fluid through the patch 20, in some implementations, inflates the patch 20 from its collapsed state.

As shown in FIG. 2, when the patch 20 is collapsed and also confined within the elongate sleeve 14, the patch 20 may be rolled up in a longitudinal direction. This helps to minimize the cross-sectional diameter of the device 100 when the patch 20 is not deployed. In one implementation described in more detail later, the rolling up of the patch 20 into the position shown in FIG. 2 occurs because the patch 20 has an internal frame that is biased to assume the rolled-up position when the patch 20 is not inflated and is urged into the confines of the sleeve 14 by moving the sleeve 14 distally with respect to the internal shaft 12.

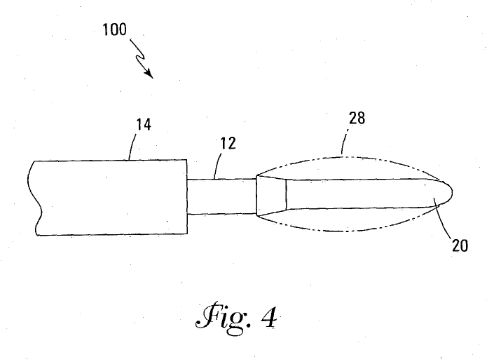

As shown in FIG. 3, the patch 20 has a balloon-like external covering 31 that forms the internal chamber 30 discussed previously. In the illustrated implementation, the patch 20, when inflated, takes on the shape of a flattened balloon appearing circular when viewed from the top (as in FIG. 3) and oval-shaped when viewed from the side (as in FIG. 4, as illustrated by dashed lines 28). More specifically, FIG. 4 shows a side view of the patch 20 shown in FIG. 3 when the patch 20 is not inflated; the dashed lines 28 indicate the degree of expansion of the patch 20 that may occur when the patch 20 is inflated. As shown in FIG. 3, the balloon-like covering 31 has a proximal opening that fits over, and attaches to, the distal end 18 of the shaft 12. Thus, the shaft's lumens 40 and 42 (shown in FIG. 3) each have openings into the internal chamber 30 of the balloon-like covering 31, thus allowing the circulation of fluid through the internal chamber 30 of the patch 20 as described previously. Instead of the circular configuration for the patch 20 as shown in FIG. 3, the patch 20 may alternatively be configured in the shape of a rectangle, square, triangle, or other suitable shape. The patch may be constructed of a thin, flexible material such as nylon, POC, PET, or other suitable materials.

Referring to FIG. 3, the patch 20 also includes an internal frame (made up of a peripheral ring 32 and longitudinal support bar 34) that provides internal structural support for the external balloon-like structure 31. The internal frame 32 and 34 causes the patch 20 to achieve a desired shape when the patch 20 is deployed. The peripheral ring 32 consists of a wire with each of its two ends attached, at attachment points 36 and 38, to the distal end 18 of the elongate shaft 12. The wire's attachment points 36 and 38 are on opposite sides of the shaft's distal end 18. From these attachment points 36 and 38, the wire 32 extends distally and in the shape of a loop that generally corresponds to the size and shape of the periphery of internal chamber 30. The longitudinal support bar 34 consists of an elongate member that has a proximal end attached, at attachment point 44, to a center portion of the shaft's distal end 18. The support bar 34 extends distally from that attachment point 44 to a distal inside surface of the external balloon-like structure 31.

The support wire 32 may be constructed of a shape-memory material, such as NITINOL, that is biased so that the wire 32 takes on both the ring configuration shown in FIG. 3 and also the rolled-up configuration shown in FIG. 2. In particular, the lateral sides of the peripheral ring 32 may be biased so that when the wire 32 is forced into the space constraints of the body's outer sleeve 14, the lateral sides of the peripheral ring fold in opposite directions so that the patch 20 rolls up into the FIG. 2 configuration. Conversely, the lateral sides of the wire 32 may also be biased outwardly so that when freed of the confines of the body's outer sleeve 14, the wire 32 takes on the ring-like configuration shown in FIG. 3.

Frame configurations other than that shown in FIG. 3 are contemplated. For example, instead of a single longitudinal support member 34 as in the FIG. 3 implementation, the patch 20 may have a plurality of similar members extending from the distal end 18 of the shaft 12 and attaching to the frame 32 in a fan-like configuration. In another configuration, there may be arms that extend radially from the member 34 to create a fishbone configuration. In other implementations, the member 34 may be omitted.

As shown in FIG. 3, the device 100 includes a temperature sensor 50 that senses temperature at a sensing element 54 located inside the chamber 30 of the patch 20. The sensing element 54 in this implementation is attached to an inside surface of the patch's balloon-like covering 31 and in a location where an outer surface of the covering 31 contacts the target tissue region. Such a placement of the sensing element 54 provides an accurate measurement of the tissue region being cooled by the device 100. In the illustrated example, the temperature sensor 50 is a thermocouple. The thermocouple is made up of two conductive wires (shown as a single wire 52 in FIG. 3 for clarity) of dissimilar materials that are insulated from each other. At a distal end that is positioned within the patch internal chamber 30, the wires are connected together to form a junction that serves as the sensing element 54. This junction 54 produces a voltage difference that is dependent on the temperature of the junction 54. The wires 52 extend proximally from the internal chamber 30, through the shaft 12 via lumen 40 and into the adapter 22, and exit the adapter 22 through the adapter port 26 (shown in FIG. 1). An external device may be connected to proximal ends of the two thermocouple wires 52 extending out of the adapter port 26 to measure the voltage difference between the wires 52, and then convert that voltage measurement into an indication of the temperature of the junction 54.

In other implementations, the temperature sensor 50 may be a thermistor or other suitable temperature sensing mechanisms. Instead of extending through lumen 40, the thermocouple wires 52 may extend through lumen 42 or through an additional lumen in the shaft 12 not shown in the illustrated implementation. Also, the temperature sensing element 54 need not be positioned on an inner surface of the balloon-like structure as shown in FIG. 3; alternatively, the temperature sensing element 54 may be positioned in other locations, either within the chamber 30 or some other location on the device 100.

With the aid of FIG. 5, a method will now be described for using the device 100 to protect myocardial tissue by cooling the tissue to reduce the injury associated with reperfusion. In FIG. 5, a distal portion of the device 100 is shown extended through an aortic valve 106 and placed inside the left ventricle 108 of a patient's heart 102. The patch 20 is shown deployed from, the body 10 of the device 100, and the patch 20 is positioned to contact a target tissue region on a region of the ventricular septum 110 that is near the heart's apex 114. Once positioned as such, cooled fluid may be provided to the patch 20 to cool the target tissue region.

To get the distal portion of the device 100 within the left ventricle 108 as shown in FIG. 5, the device 100 is first placed in the non-deployed state, as shown in FIG. 2. The distal end 15 of the device 100 may then be introduced percutaneously into the body and into an accessible vessel, such as the femoral artery, that provides access to the patient's aorta 104. The device 100 is advanced through the patient's aorta 104 and to the aortic valve 106. The aortic valve 106 opens only in the direction that allows blood to pass from the left ventricle 108 and into the aorta 104, which is the direction opposite to the direction that the device 100 is advanced through the valve 106. Thus, the device 100 may be advanced into the left ventricle 108 only when the aortic valve 106 is forced into an open position during systole (contraction). The advancement of the device 100 may be achieved by positioning the device 100 at the valve 106 and advancing it when the resistive force of the blood flow is diminished. Alternatively, the advancement of the device 100 may be achieved by monitoring the patient's electrocardiogram or a pressure indication on a physiological monitor during the procedure to determine when the aortic valve 106 is open.

Once the distal end 15 of the device 100 is advanced into the left ventricle 108, the device's distal end 15 is directed toward the ventricular septum 110. The distal end 15 may be advanced further into the ventricle 108 along the septum wall 112, and to the target tissue area, which in this example is a tissue region on the septum wall 112 near the apex 114 of the heart 102. By using the septum wall 112 as a guide and avoiding other areas within the left ventricle 108, damage to chordae tendonae or papillary tissue within the left ventricle 108 may be avoided. The movement of the patch through the ventricle 108 may also be aided by radiopaque markers (not shown) on the patch. Once the distal end 15 of the device 100 is positioned near the target tissue area, the patch 20 may be deployed by moving the sleeve 14 in a proximal direction relative to the shaft 12 (not shown in FIG. 5). The deployed patch 20 may then be placed in contact with the target tissue region.

Blood that enters the left ventricle 108 from the left atrium (not shown) applies a downward force onto the surface of the septum wall 112. As such, when the patch 20 is deployed on the septum wall 112, the blood may push the patch 20 against the wall 112. This downward force may be used to aid in the positioning of the patch 20 over the target tissue region. The patch 20 may be of sufficient flexibility to allow the patch surface to flex and conform to the septum wall 112, which increases the amount of tissue surface area in contact with the surface of the patch 20. Once the patch surface is in contact with the target tissue region, cooled fluid may be delivered to the patch 20 to cool the tissue region.

The patch 20 may also be positioned on the anterior wall of the left ventricle 108 or positioned to contact both the anterior and septal walls. These positions provide good access to the regions of the heart that are susceptible to major myocardial infarctions when blood flow is reduced. Other positions within the left ventricle 108 may also provide beneficial cooling. The useful cooling positions are dictated by the location of the papillary muscles and the chordae in the ventricle 108 and the size of the patch 20 used in the procedure. For example, in the implementation where the patch 20 is positioned on the septum wall 108, the patch 20 is approximately 2 cm by 4 cm.

The above-described procedure may be performed during a time that there is insufficient blood flow to the heart, and before and during a procedure to remove a blockage to blood flow to the heart. A procedure to remove a blockage to blood flow may be, for example, a procedure to dissolve thrombotic material in a vessel using osteolytic therapy and direct percutaneous transluminal coronary angioplasty (PCTA) procedure and an accompanying implantation of a stent. Because of the targeted cooling of the myocardial tissue susceptible to reperfusion damage using the method described above, when normal blood flow resumes and blood reperfuses into the effected areas of the heart, reperfusion injury to the myocardium will be minimized or eliminated.

This cooling method may also be used in other portions of the heart, such as the right ventricle. Cooling could also be performed in other hollow organs of the body, such as the bladder, and the intestinal tract.

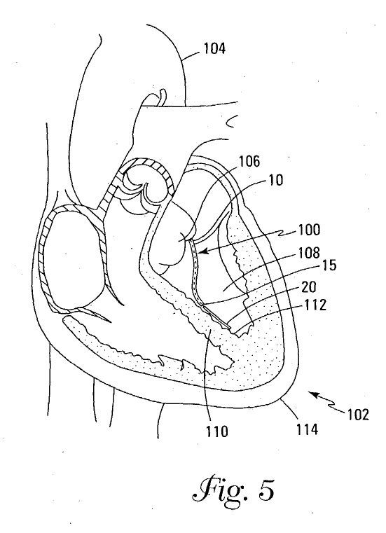

FIGS. 6-8 show a medical device 200 with a deployable structure of a different design. Referring to FIG. 6, the medical device 200 includes an elongate body 210 and a cup-shaped structure 220 that is deployable from a distal end 215 of the body 210 to cool a targeted region of internal body tissue. FIG. 6 shows the entire device 200 in a deployed state. To cool the target tissue region, the device 200 is inserted into a body lumen in a non-deployed state (not shown) and advanced until the device's distal end 215 is positioned near the target tissue region, such as inside a heart chamber. Once the device's distal end 215 is so positioned, the cup-shaped structure 220 may be deployed from the elongate body 210 and placed over a target tissue region with the open side of the cup-like structure 220 adjacent to the target tissue region. In other words, a rim structure 230 of the cup-like structure 220 is placed in contact with the target tissue region. This placement of the cup-like structure 220 creates a sealed chamber whose bounds are defined by the target tissue region and the inside of the cup-like structure 220. Cooled fluid may then be introduced into the device 220 and circulated through this sealed chamber so that the cooled fluid directly contacts and cools the target tissue region.

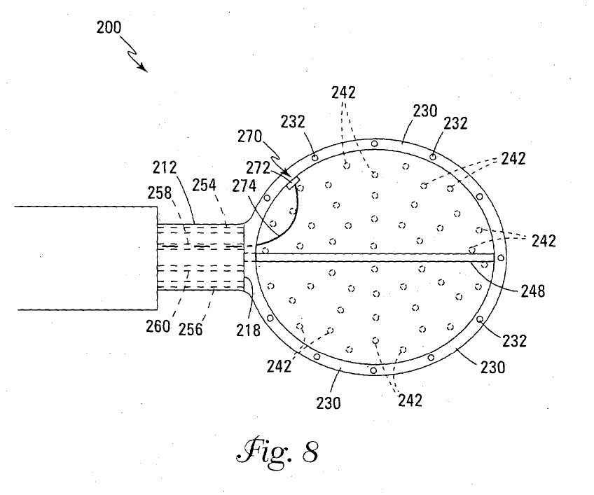

Referring to FIG. 6, the device's body 210 includes an elongate shaft 212 and an elongate sleeve 214 that encompasses the shaft 212. The cup-like structure 220 is attached to a distal end 218 of the shaft 212 (the distal end 218 being shown in FIG. 8). The elongate sleeve 214 is movable longitudinally and independently with respect to the shaft 212. FIG. 6 shows the cup-like structure 220 in a deployed state where the sleeve 214 has been moved proximally with respect to the shaft 212 so as to reveal the cup-like structure 220 from the confines of the sleeve 214. To put the cup-like structure 220 in the non-deployed state, the sleeve 214 may be pushed distally with respect to the shaft 212 so that the cup-like structure 220 collapses to fit within the sleeve 214. In the non-deployed state, the device 200 may be advanced through vessels.

An adapter 222 is attached to a proximal end 216 of the shaft 212, and remains outside of the patient's body when the device 200 is in use. Lumens 258 and 260 (a distal portion of which are shown in FIG. 8) extend longitudinally through the shaft 212 from the proximal end 216 to the distal end 218. The lumens 258 and 260 deliver fluid to and remove fluid from the cup-like structure 220, as will be described later. The cooled fluid that is circulated through the sealed chamber may be introduced through an adapter port 224 and the shaft lumen 258, which at a distal end opens into the sealed chamber formed in part by the cup-like structure 220. The fluid then exits the sealed chamber through shaft lumen 260 and adapter port 226. The fluid may be saline, blood, a blood substitute, or any other fluid with the body tissue being contacted. The temperature of the fluid provided may be, for example, five to ten degrees Celsius.

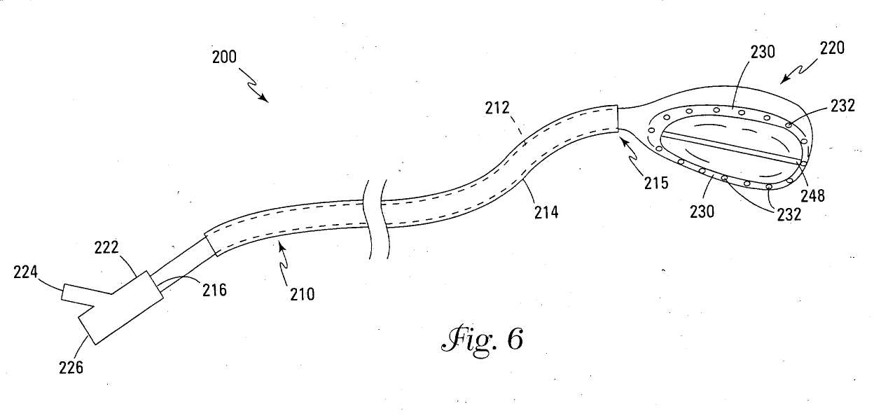

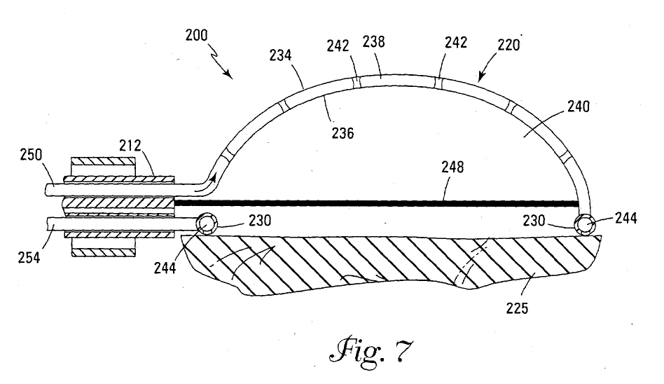

The rim structure 230 that is placed over the target tissue region includes several holes 232 on a bottom surface of the rim structure 230 which contact the surface of the tissue. These holes 232, also shown in FIG. 8, are openings to an internal channel 244 (shown in FIG. 7) within the rim structure 230 and from there to shaft lumens 256 and 258 and the adapter 222. A vacuum system may be connected to the adapter 222 to provide suction at the holes 232 so that a seal is formed between the rim structure 230 and the target tissue region.

FIG. 7 shows a cross-section of a distal portion of the medical device 200. The cup-like structure 220 is shaped like the top half of a sphere and has an outer layer 234, an inner layer 236, and an vacant chamber 238 interposed between the two layers 234 and 236. The elongate shaft 212 includes a lumen 250 that provides an inflation medium—such as a liquid or gas—to the vacant chamber 238. By filling the vacant chamber 238 with an inflation medium, the cup-like structure 220 insulates body fluids that contact the outer layer 234 from the cooled fluid that is provided to a sealed chamber 240. This insulation may limit the systemic cooling effects of the device and improve the efficiency of the targeted cooling. The inflation medium may be provided, for example, at body temperature or at any temperature suitable to insulate the body fluids.

Within the vacant chamber 238, several struts 242 are interposed between the two layers 234 and 236. The struts 242 keep the two layers 234 and 236 separated so that an inflation medium may be provided to chamber 238. In addition, the struts 242 prevent the inner layer 236 from collapsing into the chamber 240 when the chamber 238 is filled with the inflation medium. The struts 242 may be placed throughout the cup-like structure 220, as illustrated in the bottom view of the device 200 shown in FIG. 8. Alternatively, the struts 242 may be omitted from the cup-like structure if the layers 234 and 236 can sufficiently retain their shape when the cup-like structure 220 is inflated.

Referring to FIG. 7, the rim structure 230 of the cup-like structure 220 is positioned along the cup-like structure's periphery. The rim structure 230 is circular in shape and is constructed of a shape-memory material, such as NITINOL, that is biased in a desired configuration. The shape-memory material allows the cup-like structure 220 to achieve a desired shape upon the cup-like structure's deployment. The rim structure 230 includes a vacuum channel 244 that extends longitudinally through the rim structure 230 and is accessible through the holes 232 on the rim's bottom surface (shown in FIG. 8). The vacuum is applied to the vacuum channel 244 via lumens 254 and 256 to create the seal between the rim structure 230 and the tissue 225. The seal prevents the cooled fluid from escaping the sealed chamber 240 and entering the bloodstream, and at the same time prevents blood from entering the chamber 240. Other methods of providing a seal may be utilized. For example, a seal may be created by cooling the rim structure 230 enough to freeze the fluid provided to the chamber 240 at the interface of the tissue surface 225 and the rim 230. Cooling the rim structure 230 in this manner may also freeze the blood located outside of the chamber 240 near the interface of tissue 225 and rim structure 230.

To monitor the temperature of the fluid inside the chamber 240, the device 200 may include a temperature sensor 270 (shown in FIG. 8) located in the sealed chamber 240 formed by the cup-like structure 220. In the FIG. 8 implementation, the temperature sensor 270 is a thermocouple that has a junction 272 and two conductive wires 274 of dissimilar materials (shown as one wire). The wires 274 extend proximally from the chamber 240, through the shaft 212 via lumen 258, lumen 260, or an additional lumen in the shaft as described previously. In other implementations, the temperature sensor 270 may be a thermistor or other suitable temperature sensing mechanisms. The temperature sensor 270 may be located on the interior of the rim structure 230 as shown in FIG. 8, near the outlet of the lumen that provides fluid to the chamber 240, or any other location suitable for sensing the temperature of the fluid in the chamber 240.

The cup-like structure 220 may also include a support member 248 that provides support and strength to the rim structure 230. In the FIG. 8 implementation, the member 248 is attached to the distal end 218 of the shaft 212 and extends longitudinally through the chamber 240 and attaches to the inside layer 236 of the cup-like structure 220. In other examples, the cup-like structure 220 may include a plurality of similar members to provide further support extending from the shaft 212 and attaching to the inside layer 236 to form a fan-like configuration. Alternatively, there may also be arms that extend radially from the member 248 to form a fishbone configuration. In other implementations, the member 248 may be omitted.

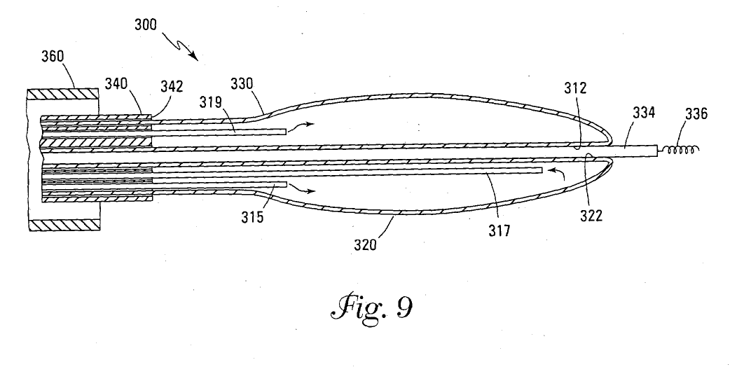

FIG. 9 shows a cross-section of a distal portion of another medical device 300 with a deployable structure of a different design. The medical device 300 shown in FIG. 9 includes a patch 320 and an insulating balloon 330 that are deployable from a distal end 342 of an elongate body. FIG. 9 shows the device 300 in a deployed state. As in previously described embodiments, the device's body includes an elongate shaft 340 and complementary elongate sleeve 360, which each function as in the previously described embodiments to deploy and retract the deployable structure, which in this example includes both the patch 320 and the insulating balloon 330. To cool a target tissue region, the patch 320 is placed against a target tissue region and cooled fluid is circulated through the patch 320 via lumens 315 and 317, as described in other implementations. The insulating balloon 330 may be attached to the patch's top surface 322 and filled with a fluid or gas via lumen 319 to insulate body fluids from the cool fluid circulating in the patch 320. The balloon may also have an additional lumen to remove the insulation medium from the insulation balloon 330 if desired.

The medical device 300 may also include a hollow shaft 334 that extends distally from the shaft 340 between the insulation balloon's bottom surface 312 and the patch's top surface 322. A wire 336 may be extended through the shaft 334 via a lumen in the medical device 300 (not shown) and into an area of tissue to anchor the patch 320 against the target tissue region. In the FIG. 9 implementation, the distal end of the wire 336 is formed in the shape of a spiral so that it can be screwed into an area of tissue. In other implementations, the wire may be formed in a variety of different ways to secure the position of the patch 320. Alternatively, the shaft 334 and the wire 336 may be omitted.

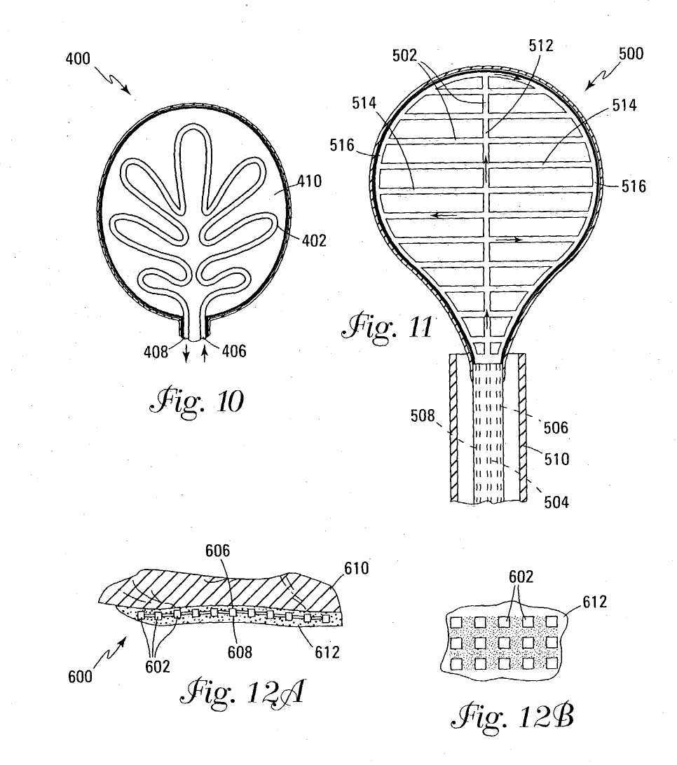

FIGS. 10 and 11 show alternative implementations of a deployable patch having flow circuits or conduits that direct cooled fluid through the patch. In the FIG. 10 implementation, the patch 400 includes a tube 402 that winds throughout a substantial portion of the patch's internal chamber 410. To cool the target tissue area, cooled fluid is circulated through the tube 402 through openings 406 and 408. In the FIG. 11 implementation, the patch 500 includes a plurality of fluid channels 502 that are constructed in a grid-like manner. Fluid may be provided through lumen 504 into the center channel 512 that extends longitudinally from the shaft 510 as indicated by the arrow. The fluid may then flow from the center channel 512 into a plurality of channels 514 that run in a direction perpendicular to the center channel 512. The fluid may then flow back toward the shaft 510 along return channels 516 and drain into the lumens 506 and 508. The patches 400 and 500 shown in FIGS. 10 and 11 are only two examples of flow circuits that could be implemented. In other implementations, the flow circuits could take on a variety of shapes and configurations.

FIGS. 12A and 12B show a portion of a patch 600 having a series of thermoelectric cooling (TEC) elements 602 to cool a target tissue region 610. The TECs 602 cool the tissue using a thermal energy process know as the Peltier effect. To utilize this process, a voltage DC power source may be applied to the element 602 to move heat through the element 602 from one side to the other. FIG. 12A is a cross-section view of a the patch 600. FIG. 12B is a top view of a portion of the patch 600 illustrating how the elements 602 are spaced throughout the patch 600.

In the FIG. 12A implementation, the elements 602 are packaged within an electric insulator 612 and include an n-type semiconductor and a p-type semiconductor connected in series (the semiconductors not being shown). In other implementations, the semiconductors may be replaced with other suitable materials. The semiconductors are arranged between a ceramic substrate that electrically insulates the conductors from heat sinks attached to the ceramic substrate on two sides 606 and 608 of the element 602 (the ceramic substrate and heat sinks not being shown). The elements 602 are arranged so that one heat sink is adjacent to a contact surface 606 and the other heat sink is adjacent to a top surface 608.

Applying the DC voltage to the elements 602 causes a current to pass through the semiconductors pairs. The current causes heat to be drawn from the heat sink near the contact surface 606 to the heat sink near the top surface 608. Through this process, the contact surface 606 is cooled, and at the same time, the top surface 608 is heated. By cooling the contact surface 606 of the patch 600, the target tissue region 610 may also be cooled.

A number of implementations have been described. Nevertheless, it will be understood that various modifications may be made. For example, the devices and methods described can be used to cool other tissue in the body, such as the right ventricle, the bladder, and the intestinal tract. Accordingly, other embodiments are within the scope of the following claims.

What is claimed is:

1. A medical device comprising:
an elongate body having a distal end for entry into a body and positionable near a target tissue region within the body; and
a structure deployable from the distal end of the elongate body to cool the target tissue region.

2. The medical device of claim 1 wherein the elongate body comprises:
an elongate shaft that has the deployable structure affixed to its distal end; and
an elongate sleeve that is longitudinally movable with respect to the shaft, wherein the elongate sleeve, when moved distally, encompasses the deployable structure.

3. The medical device of claim 1 wherein the deployable structure comprises a patch having a surface shaped to contact the tissue region.

4. The medical device of claim 3 wherein the deployable patch has an inner chamber that receives, from a lumen in the elongate body, a fluid for cooling the patch surface that contacts the target tissue region.

5. The medical device of claim 1 wherein the deployable structure is cup-shaped and has a periphery for contacting body tissue to form a chamber whose bounds are defined by the body tissue and an inside surface of the cup-shaped structure.

6. The medical device of claim 1 wherein the deployable structure has an inner chamber with a Joule-Thompson orifice into the inner chamber so that a liquid supplied through the-elongate body, through the orifice, and into the inner chamber has a phase change into a gas.

7. The medical device of claim 1 wherein the distal end of the device is advanceable through a body vessel to the target tissue region when the structure is in a non-deployed state.

8. The medical device of claim 7 wherein the structure cannot be advanced through the body vessel when the structure is in a deployed state.

9. The medical device of claim 1 wherein the elongate body further comprises a proximal end that remains outside the body when the distal end of the elongate body is positioned near the target tissue region.

10. A medical device comprising:
an elongate body having a distal end for entry into a body and positionable near a target tissue region within the body;
a patch deployable from the distal end of the elongate shaft to cool the target tissue region, the patch having a surface shaped to contact the target tissue region.

11. The medical device of claim 10 wherein the patch comprises a collapsible frame made of a shape memory alloy so that, when deployed, the patch expands to create the surface that contacts the target tissue region.

12. The medical device of claim 10 wherein the elongate body comprises:
an elongate shaft that has the deployable patch affixed to its distal end; and
an elongate sleeve that is longitudinally movable with respect to the shaft, wherein the elongate sleeve, when moved distally, encompasses the deployable patch.

13. The medical device of claim 12 wherein the patch is deployed from the distal end of the body by moving the sleeve proximally with respect to the shaft to expose the patch from the confines of the sleeve.

14. The medical device of claim 10 wherein the distal end of the device is advanceable through a body vessel to the tissue region when the patch is in a non-deployed state.

15. The medical device of claim 14 wherein the distal end of the device is not advanceable through the body vessel when the patch is in a deployed state.

16. The medical device of claim 10 wherein the elongate shaft comprises:
a first lumen to provide fluid to an inner chamber of the patch; and
a second lumen to remove fluid from the inner chamber of the patch.

17. The medical device of claim 16 wherein the inner chamber of the patch comprises a conduit through which fluid flows, the conduit being located adjacent to the surface of the patch in contact with the target tissue region.

18. The medical device of claim 10 further comprising at least one additional patch deployable from the distal end of the elongate body.

19. The medical device of claim 10 wherein the patch comprises a thermoelectric cooling element positioned adjacent to the surface of the patch and in contact with the target tissue region that cools the target tissue region.

20. The medical device of claim 10 further comprising a balloon positioned adjacent to a surface of the patch that does not contact the target tissue region, the balloon providing insulation between the patch and body fluids when the patch is deployed and positioned near the target tissue region.

21. The medical device of claim 20 wherein the elongate body comprises a lumen to provide the balloon with an inflation medium.

22. The medical device of claim 20 further comprising an anchoring mechanism near the distal end of the elongate shaft that is connectable to tissues inside the body to anchor the patch when it is deployed near the target tissue area.

23. The medical device of claim 10 wherein the patch has an inner chamber with a Joule-Thompson orifice into the inner chamber so that a liquid supplied through the elongate shaft, through the orifice, and into the inner chamber has a phase change into a gas.

24. The medical device of claim 10 wherein the patch comprises a temperature sensor to sense the temperature of the surface of the patch in contact with the target tissue region.

25. A medical device comprising:
an elongate body having fluid transfer lumens extending longitudinally through the shaft to a distal end; and
a cup-shaped structure deployable from the distal end of the body and having a periphery for contacting body tissue to form a chamber bound by the body tissue and an inside surface of the cup-shaped structure, the chamber being in fluid communication with the body fluid transfer lumens to allow fluid to be delivered to and from the chamber.

26. The medical device of claim 25 wherein the cup-shaped structure comprises a collapsible frame made of a shape memory alloy so that, when deployed, the cup-shaped structure can expand to take on a cup-shaped configuration.

27. The medical device of claim 25 wherein the elongate body comprises:
an elongate shaft that has the deployable structure affixed to its distal end; and
an elongate sleeve that is longitudinally movable with respect to the shaft, wherein the elongate sleeve, when moved distally, encompasses the deployable structure.

28. The medical device of claim 27 wherein the cup-shaped structure is deployed from the distal end of the body by moving the distal end of the sleeve proximally with respect to the shaft to expose the cup-shaped structure from-the confines of the sleeve.

29. The medical device of claim 25 wherein the distal end of the device is advanceable through a body vessel to the tissue region when the cup-shaped structure is in a non-deployed state.

30. The medical device of claim 29 wherein the distal end of the device is not advanceable through the body vessel when the cup-shaped structure is in a deployed state.

31. The medical device of claim 25 wherein the periphery of the cup-shaped structure comprises small holes positioned to contact the body tissue, the holes securing the periphery of the cup-shaped structure to the body tissue when a vacuum is applied.

32. The device of claim 25 wherein the cup-shaped structure comprises a layered structure with an inflatable chamber positioned between first and second layers and inflatable to insulate body fluids from the fluid in the sealed chamber.

33. The device of claim 32 wherein the elongate shaft comprises a lumen to provide the inflatable chamber with an inflation medium.

34. The device of claim 25 further comprising a temperature sensor to sense the temperature of the fluid delivered to and from the chamber.

35. A method of cooling a target tissue region inside a body, the method comprising:
introducing into a body vessel a distal portion of a catheter having an elongate body and a structure deployable from a distal end of the elongate body;
positioning the distal portion of the catheter near the target tissue region;
deploying the deployable structure from the elongate body;
placing the deployed structure in contact with the target tissue region; and
cooling the deployed structure to cool the target tissue region.

36. The method of claim 35 wherein the deployable structure comprises a patch having a surface shaped to contact the tissue region.

37. The method of claim 35 wherein the deployable structure is cup-shaped and has a periphery for contacting to the body tissue region to form a chamber bound by the body tissue and an inside surface of the cup.

38. The method of claim 35 wherein the target tissue region is within a chamber of the heart.

39. The method of claim 38 wherein the deploying of the deployable structure occurs after the distal end of the catheter is positioned inside the chamber of the heart.
